(19) United States
(12) Patent Application Publication
(10) Pub. No.: US 2024/0131060 A1
(43) Pub. Date: Apr. 25, 2024

(54) PATIENT SELECTION FOR TREATMENT WITH DENDRITIC CELL VACCINATION

(71) Applicant: SCTbio a.s., Prague 7 (CZ)

(72) Inventors: Jitka PALICH FUCIKOVA, Roztoky (CZ); Radek SPISEK, Prague 5 (CZ)

(21) Appl. No.: 18/277,041

(22) PCT Filed: Feb. 11, 2022
(86) PCT No.: PCT/EP2022/053356

§ 371 Date: Aug. 10, 2023

(30) Foreign Application Priority Data
Feb. 12, 2021 (EP) 21156839.9
Mar. 17, 2021 (EP) 21163348.2

(51) Int. Cl. A61K 35/15; A61K 39/00; A61K 45/06; A61P 35/00; C07K 14/705; C12N 5/0784; C12Q 1/6886; G01N 33/574

(52) U.S. Cl. CPC A61K 35/15; A61K 39/4615; A61K 39/464402; A61K 45/06; A61P 35/00; C07K 14/705; C12N 5/0639; C12Q 1/6886; G01N 33/574

(57) ABSTRACT

The invention relates to a dendritic cell (DC) vaccine for use in a method of treating cancer in a patient, wherein the patient is selected for treatment with said DC vaccine. The selection inter alia comprises determining in a tumor sample from the patient the amount of CD8+ T-cells and/or the tumor mutation burden (TMB) and comparing the determined amount to a predetermined threshold level. The selection allows identifying patients that particularly benefit from DC treatment.

The immune system is a powerful tool that, if better harnessed, could enhance the efficacy of cytotoxic agents and improve outcomes for cancer sufferers. Vaccination is hoped to be an effective method of harnessing the immune system to eliminate cancer cells. Its activity is mostly dependent on antigen-specific CD8⁺ T cells that generate cytotoxic T lymphocytes (CTLs) to reject cancer (Palucka and Banchereau 2013). The immune system components necessary for the induction of such CD8⁺ T cells include the presentation of antigens by appropriate antigen-presenting cells (APCs). Dendritic cells (DCs) are the most efficient APCs, and are an essential component of vaccination through their capacity to capture, process, and present antigens to T cells (Banchereau and Steinman 1998). Multiple cancer vaccination strategies based on DCs have been developed (Galluzzi et al. 2012). One strategy is to culture patient-derived (autologous) monocytes with specific cytokine combinations, load them with tumor-associated antigen (TAAs) ex-vivo in the presence of adjuvants to promote DC maturation and eventually re-administer them into the patient. Other strategies are to deliver TAAs to DCs in vivo or approaches based on DC-derived exosomes (Galluzzi et al. 2012). These strategies have been tested in multiple clinical trials, ranging over a large variety of cancers (Galluzzi et al. 2012; Vacchelli et al. 2013; Bloy et al. 2014; Garg et al. 2017). A newly developed DC vaccine, based on dendritic cell obtained from patient-derived (autologous) monocytes loaded with tumor cells undergoing immunogenic cell death (ICD) and matured with Toll-like receptor (TLR) agonists binding TLR3 or TLR4 has been shown to be more beneficial for the overall survival (OS) and progression free survival (PFS) of ovarian cancer patients than standard of care chemotherapy alone (Cibula et al. 2018; Rob et al. 2018).

Efficacy of such a DC vaccine treatment may further be improved by identifying, prior to treatment, patients who would benefit from such treatment and there is still a need to improve DC vaccine treatment outcome especially for cancer patients with a bad prognosis.

It is now well established that most cancers have evaded immune attacks when diagnosed, whereas residual signs of an active anticancer immune response within the tumor usually indicate a positive prognosis. Positive immune-related prognostic features include the presence of specific T-cells subsets in the tumor, often CD8⁺ T-cells, the absence of immunosuppressive elements, as well as the localization and specific organization of the immune infiltrate. Likewise, the absence or low presence of such residual active anticancer immune response may be interpreted as an indicator of a bad prognosis. The presence of high tumor mutational burden (TMB) has also been identified as a positive prognostic feature. Altogether, the presence of tumor-infiltrating CD8⁺ T-cells and high TMB has often been used to define hot tumors, while cold tumors have been defined as having low or absent tumor-infiltrating CD8⁺ T-cells and low TMB (Maleki Vareki 2018).

The inventors have now surprisingly found that patients with a low amount of infiltrating CD8⁺ T-cells in the tumor and/or low TMB, or altogether indicative of a cold tumor, typically diagnosed with a bad prognosis, had an improved survival rate when treated with a dendritic cell vaccine, when compared to patients treated with the standard of care chemotherapy only. In comparison, patients with a high amount of infiltrating CD8⁺ T-cells in the tumor and/or high TMB did not significantly benefit from the DC vaccination compared to patients treated with standard of care chemotherapy only. Accordingly, the inventors identified CD8⁺ T-cells and/or TMB as a predictive marker for treatment success for dendritic vaccines and a respective threshold of the amount of CD8⁺ T-cells and/or TMB in a tumor sample allowing to select patients which will greatly benefit from a treatment with dendritic cell vaccines.

### Definitions

“Dendritic cell vaccine” or “DC vaccine” refers to human DCs for therapeutic use that may be administered, being prepared without an antigen source or with an antigen source. Preferably, the DCs have been prepared by loading the DCs with an antigen sourced from tumor associated peptide(s), whole antigens from DNA or RNA, whole antigen-protein, idiotype protein, tumor lysate, whole tumor cells or viral vector-delivered whole antigen. The loaded DCs may be matured with toll-like receptor agonists. In a preferred embodiment, the DCs have been loaded with whole tumor cells undergoing immunogenic cell death as described for example by Fucikova et al. (2014). Further, the loaded DCs may be further matured. Maturation occurs e.g. by culturing the loaded DCs in the presence of maturation factors such as Toll-like receptor agonists (e.g., poly[I:C] or LPS).

“Toll-like receptor agonists” refers to molecules binding toll-like receptors (TLR), e.g. lipopolysaccharides binding to TLR4, double-stranded RNA or polyinosinic:polycytidylic acid (poly[I:C]) binding to TLR3. Further, TLR1, TLR2, TLR5 and TLR8 agonists are suitable for maturation. TLR expression and function in DCs is reviewed by Schreibelt et al. (2010), see table 1 for monocyte derived DCs (moDC).

“Chemotherapy” is a treatment that uses drugs or agents to stop the growth of cancer cells, either by killing the cells (cytotoxic agents) or by stopping them from dividing (cytostatic agents). Chemotherapy drugs may include alkylating agents or alkylating-like agents such as carboplatin or cisplatin, antimetabolites such as gemcitabine, pemetrexed, methotrexate, anti-tumor antibiotics such as doxorubicin, topoisomerase inhibitors such as topotecan, irinotecan or etoposide, mitotic inhibitors such as docetaxel, paclitaxel, vinblastine, or vinorelbine. Further examples of chemotherapy drugs are provided by the American Cancer Society and can be found here: https://www.cancer.org/treatment/treatments-and-side-effects/treatment-types/chemotherapy/how-chemotherapy-drugs-work.html (‘American Cancer Society—How Chemotherapy Drugs Work’ 2016). Such drugs may typically be small molecule drugs (organic compounds of low molecular weight, e.g. <900 Dalton). Chemotherapy may also include, as combination partners, other treatments such as hormones and hormone analogous, small molecules drugs (e.g. kinase inhibitors, PARP inhibitors), vaccines other than DC vaccines, or biologics (e.g. antibodies), or combinations thereof. Chemotherapy may be given orally, by injection, or infusion, or on the skin, depending on the type and stage of the cancer being treated. It may be given alone or with other treatments, such as surgery, radiation therapy.

The term chemotherapy also covers “neoadjuvant chemotherapy”, which is a chemotherapy treatment given as a first step to shrink a tumor before the main treatment, which is usually surgery.

The term chemotherapy also covers “adjuvant chemotherapy”, which is a chemotherapy treatment given after the primary treatment (for example surgery or radiation) to treat residual tumor and/or to prevent or treat metastasis. In the context of the present invention, dendritic cell vaccines are considered not to be adjuvant chemotherapy.

“First-line therapy” is a first treatment given for a disease. It is often part of a standard set of treatments, such as surgery followed by chemotherapy and radiation. When used by itself, first-line therapy is typically the one accepted as the best treatment for a disease. If it does not cure the disease or if it causes severe side effects, other treatment options may be added or used instead. The first-line therapy can also be called induction therapy, primary therapy, or primary treatment.

“First-line chemotherapy” is a therapy with at least one chemotherapeutic agent as part of a first-line therapy.

“Second-line chemotherapy” is a chemotherapy that is given when the patient does not respond to the initial treatment (first-line therapy), or if the first-line therapy stops being effective. “Third-line chemotherapy” is the chemotherapy that is given when both initial treatment (first-line therapy) and subsequent treatment (second-line therapy) were not effective or stopped being effective.

“Completion” of chemotherapy is achieved when the last administration of the chemotherapy within the last cycle is completed. For the avoidance of doubt, the last cycle may be the last scheduled cycle or, if chemotherapy is terminated (e.g. prior to completion due to undesired side effects or non-response to the treatment), it is the last administered cycle.

“Maintenance therapy” or “maintenance treatment” is a treatment following the initial chemotherapy or starting within the initial chemotherapy after complete remission or partial response (Sakarya 2016; Ellis 2016). It is given to help keeping cancer from coming back after it has disappeared or shrunk. A treated cancer may “relapse” or be “recurrent” when the cancer or signs and symptoms of the cancer return after a period of improvement.

“Overall survival” (OS) is the length of time from either the date of diagnosis, date of randomization or the start of treatment for a disease, such as cancer, that patients diagnosed with the disease are still alive. In a clinical trial, measuring the overall survival is one way to measure the efficacy of a new treatment. Randomization into a clinical trial is the process by which subjects are assigned by chance to separate groups that compare different treatments or other interventions. Randomization gives each participant an equal chance of being assigned to any of the groups.

“Overall survival rate” is the percentage of people in a study or treatment group who are still alive for a certain period of time after they were diagnosed with a disease, randomized or started treatment for a disease, such as cancer. The overall survival rate is often stated as a five-year survival rate, which is the percentage of people in a study or treatment group who are alive five years after their diagnosis or the start of treatment.

“Progression-free survival (PFS)” is the length of time during and after the treatment of a cancer, that a patient lives with the disease but it does not get worse.

“Standard of care” (SOC) is a treatment that is accepted by medical experts as a proper treatment for a certain type of disease and that is widely used by healthcare professionals. It can also be called best practice, standard medical care, and standard therapy.

“Predetermined threshold level” refers to a threshold level that has been set, e.g., by the treating physician or that is specified in the product leaflet. The predetermined threshold level may universally apply to all patients. Alternatively, different threshold levels may apply to different patient groups depending on their age, history of disease, co-morbidities, tumor, sex, etc.

“CD8⁺ T cell” (also known as cytotoxic T cell, T_{C}, cytotoxic T lymphocyte, CTL, T-killer cell, cytolytic T cells or killer T cell) is a T lymphocyte expressing the protein cluster of differentiation 8 (CD8) that is able to kill cancer cells, infected cells or cells that are damaged in other ways.

A “cold tumor” is a tumor with a low amount or absence of lymphocytes, such as CD3⁺ T-cells and/or CD8⁺ T-cells, within the tumor and at the tumor edges. A cold tumor may also exhibit a low TMB. It may also be a tumor with failed T cell priming which has a low tumor mutational burden (TMB). It may show low expression of antigen presentation machinery markers such as major histocompatibility complex class I (MHCI). It may also exhibit an intrinsic insensitivity to T cell killing. A cold tumor may also be a tumor that has not triggered a strong immune response, and/or a tumor which doesn't show signs of inflammation.

When the term “about” is used herein in relation to numerical values, it is understood as being ±5%, preferably ±2%, of the numerical value.

Technical terms are used by their common sense. If a specific meaning is conveyed to certain terms, definitions of terms will be given in the following in the context of which the terms are used.

### DESCRIPTION OF THE INVENTION

In a first aspect the invention relates to a dendritic cell (DC) vaccine for use in a method of treating a tumor in a patient, wherein the patient is selected for treatment with said dendritic cell vaccine, the selection comprising determining from a tumor sample of the patient that the tumor is a cold tumor.

In another aspect, the invention relates to a dendritic cell vaccine for use in a method of treating a tumor in a patient, wherein the patient is selected for treatment with said dendritic cell vaccine, the selection comprising determining that the tumor exhibits a low amount or the absence of infiltrating CD8⁺ T-cells. A low amount of infiltrating CD8⁺ T-cells is understood corresponding to an amount of CD8⁺ T-cells in the tumor which is below the median amount reported for the type of tumor/cancer to be treated, when quantified by immunohistochemistry on tumor samples, as described in Steele, 2018 (Steele et al. 2018). FIG. 4 of Steele, 2018 and the method to evaluate the density of infiltrating CD8⁺ T-cells are hereby incorporated by reference. FIG. 4 of Steele shows a median of infiltrating CD8⁺ T-cells of about 120 cells/mm² for renal cell carcinoma (RCC), about 130 cells/mm² for urothelial bladder carcinoma (UBC), about 210 cells/mm² for gastroesophageal carcinoma (GEC), about 250 cells/mm² for head & neck squamous cell carcinoma (HNSCC), about 250 cells/mm² for non-squamous non-small cell lung cancer (LNSQ), and about 310 cells/mm² for pancreatic carcinoma (PANC). In one embodiment, the method comprises determining the number of CD8⁺ T cells in the tumor sample and selecting the patient for treatment when the number of CD8+ T cells is below the median. The cancer to be treated may correspond to a type of cancer were the measured amounts of infiltrating CD8⁺ T-cells are distributed over about two orders of magnitude over a sampled population and/or where the median amount is not above 350 CD8⁺ T-cells/mm². Accordingly, the DC vaccine may be for the treatment of cancer types showing a high variation of tumor infiltrating CD8⁺ T cell levels. Among patients suffering from those cancer types, patients exhibiting low levels of tumor infiltrating CD8⁺ T cells particularly benefit from DC vaccine treatment.

In another preferred embodiment, a low amount of infiltrating CD8⁺ T-cells is understood corresponding to an amount of CD8⁺ T-cells in the tumor which is within the lower tertial of the amount of infiltrating CD8⁺ T cells reported for the type of tumor/cancer to be treated, when quantified by immunohistochemistry on tumor samples (Steele et al. 2018).

When the type of tumor/cancer to be treated has not been previously characterized for tumor infiltrating CD8⁺ T-cells, the patient may be selected once the tumor infiltrating CD8⁺ T-cells characterization has been established based on multiple tumor samples similarly to Steele 2018, the patient being selected when having an amount of infiltrating CD8⁺ T cells below the median or within the lower tertial.

In a preferred aspect the invention relates to a dendritic cell (DC) vaccine for use in a method of treating a tumor in a patient, wherein the patient is selected for treatment with said dendritic cell vaccine, the selection comprising determining in a tumor sample from the patient the amount of CD8⁺ T-cells and comparing the determined amount to a predetermined threshold level.

Immunological components of the tumor microenvironment have been attributed robust diagnostic and/or predictive value in multiple cohorts with cancer (Fridman et al. 2017). In a large variety of cancer types, high levels of tumor-infiltrating CD8⁺ T-cells are strongly associated with prolonged survival in cancer patients. Likewise, the presence of tertiary lymphoid structures (TLS) and macrophages of the M1 subtype is associated with prolonged survival on patients in specific types of cancer. To the contrary, the presence of regulatory T cells (Treg cells) and macrophages of the M2 subtype is generally associated with bad prognosis (Fridman et al. 2017).

The inventors now found in a clinical study of patients with newly diagnosed epithelial ovarian cancer (Clinical trial information: NCT02107937) that those patients treated with a dendritic cell vaccine in combination with standard of care chemotherapy having a low CD8⁺ T-cell count in tumor samples had a significantly improved clinical outcome compared to patients treated with the chemotherapy alone, both with respect to progression free survival and overall survival. However, such effect could not be observed for those patients from the same study which had high CD8+ T-cell counts in their tumor samples. This finding is surprising as, as stated above, patients with low tumor infiltrating lymphocytes of the so-called “cold” tumor phenotype, are seen as the most challenging to treat (Galon and Bruni 2019). Cold tumors are typically defined as tumors with a low Immunoscore (Immunoscore I0/non-inflamed) (see FIG. 1 and page 197, last paragraph to page 198, first paragraph of Galon, 2019, supra).

In another aspect, the selection of the patient may also comprise determining in a tumor sample from the patient the amount of tertiary lymphoid structures (TLS) and comparing it to a predetermined threshold level. A marker of TLS is the presence of cell clusters composed of DC-Lamp⁺ mature dendritic cells within a tumor, and may be visualized and the amount quantified as disclosed in Goc et al. (2014).

In yet another aspect, the selection of the patient may also comprise determining in a tumor sample from the patient the amount of macrophages of the M1 subtype and comparing it to a predetermined threshold level. Expression of iNOS has been identified as a marker of macrophages of the M1 subtype, and may be used in immunostaining of a tumor sample for the visualization and determination of the amount of this type of macrophage, as disclosed in Lisi et al. (2017).

It is understood that the tumor-infiltrating cell type selected to determine the threshold may depend on the type of cancer to be treated. For example, the presence of CD8⁺ T cells and TLS may be tested for selecting a patient suffering from breast cancer, melanoma, pancreatic cancer, non-small cell lung cancer (NSCLC), gastric cancer or head and neck cancer. In the case of NSCLC and gastric cancer, selection marker may also comprise the presence of macrophages of the M1 subtype. For ovarian cancer and hepatocellular cancer, selection marker may comprise presence of CD8⁺ T cells and macrophages of the M1 subtype.

In another embodiment of the invention, the invention relates to a dendritic cell (DC) vaccine for use in a method of treating cancer in a patient, wherein the patient is selected for treatment with said dendritic cell vaccine, the selection comprising determining in a tumor sample from the patient the tumor mutation burden (TMB) and comparing the determined TMB to a predetermined threshold level. The TMB may be expressed in mutation/megabase (mut/Mb), wherein the mutation may be somatic, coding, base replacement, and insert-deletion mutations in the genome from the DNA extracted from a tumor sample. The tumor sample may be a fresh, frozen or fixed tumor sample. TMB may be evaluated in a tumor sample as disclosed in Buttner et al (Buttner et al. 2019) by next generation sequencing (Head 2018). Clinical studies to dissect the genetic makeup of tumors revealed that patients with high TMB have increased response rates and improved outcomes to treatment with immunotherapy compared with patients with lower TMB (Buttner et al. 2019). The inventors have now found in a clinical study of patients with newly diagnosed epithelial ovarian cancer (Clinical trial information: NCT02107937) that those patients treated with a DC vaccine in combination with standard of care chemotherapy having a low TMB had a significantly improved clinical outcome compared to patients having a low TMB treated with the chemotherapy alone. However, such effect could not be observed for those patients from the same study which had a high TMB.

It has been reported that cold tumor, defined by low tumor infiltrating lymphocytes, had often also low TMB, and “hot” tumors (having high tumor infiltrating lymphocytes such as CD8⁺ T-cells) had a high TMB (Maleki Vareki 2018). The finding that patient having a cold tumor, defined as low CD8⁺ T-cells infiltration and/or a low TMB, had a significantly improved clinical outcome when treated with a DC vaccine is thus surprising as patients with cold tumor/low TMB are seen as the most challenging to treat.

In one embodiment, the invention relates to a dendritic cell (DC) vaccine for use in a method of treating cancer in a patient, wherein the patient is selected for treatment with said dendritic cell vaccine, the selection comprising determining in a tumor sample from the patient whether the tumor is a cold tumor. In one embodiment, determining a cold tumor may comprise determining in the tumor sample from the patient the amount of CD8⁺ T-cells and comparing the determined amount to a predetermined threshold level. In another embodiment, determining a cold tumor may comprise determining in the tumor sample from the patient the tumor mutation burden (TMB) and comparing the determined TMB to a predetermined threshold level. In yet another embodiment, determining a cold tumor may comprise determining in the tumor sample from the patient the tumor mutation burden (TMB) and comparing the determined TMB to a predetermined threshold level and determining in the tumor sample from the patient the amount of CD8⁺ T-cells and comparing the determined amount to a predetermined threshold level. The inventors have shown that tumors with low amount of CD8⁺ T-cells are also tumors with low TMB (see FIG. 6).

In one embodiment, the patient is selected for treatment in case the determined amount of CD8⁺ T-cells is equal to or below the predetermined threshold level and/or in case the determined TMB is equal to or below the predetermined threshold level.

In one embodiment of the invention, the amount of CD8⁺ T-cells is measured in a tumor sample. Preferably, the amount of CD8⁺ T-cells is measured from at least one section of the tumor sample. The section may have a thickness between 2 and 4 μm. It is understood that the tumor sample is from the patient to be treated and that the tumor sample has been prepared according to standard procedures of histology, as disclosed e.g. in Dey (2018). The tissue may be fixed for 24 hours in a 10% neutral buffered formalin, followed by embedding in paraffin. The smaller the size of the tissue, the better is the infiltration of the embedding medium. The paraffin-embedded tumor may then be cut in sections having 2 to 4 μm of thickness, preferably 3 μm of thickness. The thickness of the section of the tumor sample should allow identification of the CD8⁺ T-cells by microscopy and standardization of the cell counts. The thickness of the section is chosen so that a on average single layer of cells is analyzed. Accordingly, the thickness of the sections should not have an impact on the amount of CD8⁺ T-cells determined.

In another embodiment, the amount of CD8⁺ T-cells is measured in a tumor sample involving dissociation of the tumor sample and FACS analysis.

In another embodiment, the predetermined threshold level of CD8⁺ T-cells in the section of the tumor sample allowing selection of patients to be treated with the dendritic cell vaccine is between 30-40 CD8⁺ T-cells/mm² (disregarding the thickness of the section). In one embodiment, the predetermined threshold level is about 40 CD8⁺ T-cells/mm², about 35 CD8⁺ T-cells/mm² and preferably about 30 CD8⁺ T-cells/mm². The amount of CD8⁺ T-cells/mm² may be counted using a regular light microscope by a trained histologist or using a histology slide scanner. When using a histology slide scanner with subsequent automated software analysis, an area of interest has to be predefined by a trained pathologist to avoid counting of cells which may not be in the tumor tissue. The amount of CD8⁺ T-cells may be counted for the whole tumor tissue section and averaged to obtain counts per mm². FIG. 5A-D shows distributions of counts of CD8⁺ T-cells in tumor tissue slides from patients selected for the first line treatment of ovarian cancer with a DC vaccine in combination with standard of care or with standard of care only.

In yet another embodiment, the patient is selected to be treated with the DC vaccine when the tumor sample from the patient contains an amount of CD8⁺ T-cells equal or below the predetermined threshold level. The inventors have now shown that patients with newly diagnosed epithelial ovarian carcinoma, who have undergone debulking surgery and treated with a DC vaccine in parallel or sequentially to standard of care, selected for an amount of CD8⁺ T-cells equal or below 30 cells/mm², had an improved progression-free survival (PFS) and overall survival (OS) when compared to patients selected for an amount of CD8⁺ T-cells equal or below 30 cells/mm² but only treated with the standard of care (see Example 3 below and FIG. 2 and FIG. 3).

In one embodiment, the patient is selected for treatment with the DC vaccine in case the patient has a TMB equal or below the predetermined threshold level.

In one embodiment, the selection of a patient involves determining the amount of CD8+ T-cells and determining the TMB.

In another aspect, the invention relates to a dendritic cell vaccine for use in a method of treating cancer in a patient, wherein the method comprises a step of determining in a tumor sample of the patient, whether or not the tumor sample contains an amount of CD8⁺ T-cells/mm² that is equal or below a predetermined threshold level. The predetermined threshold level may be between 30-40 CD8⁺ T-cells/mm². In one embodiment, the predetermined threshold level is about 40 CD8⁺ T-cells/mm², preferably about 35 CD8⁺ T-cells/mm² and especially about 30 CD8⁺ T-cells/mm². As cancer treatment often involves a first step of removing most of the tumor mass by debulking surgery, or taking biopsies, a tumor sample of the patient is usually available for evaluation. The tumor sample is usually processed for histology and pathology analysis, implying fixation of the tumor sample, sectioning of the tumor sample and immunocytochemistry or immunohistochemistry. The basic principles of such tissue processing are disclosed in Dey (2018). Determining the amount of CD8⁺ T-cells/mm² may involve the use of antibodies during immunocytochemistry or immunohistochemistry targeting CD8⁺ T-cells. Such antibodies are well known in the field and are exemplified in Example 2 below. Counting the CD8⁺ T-cells in the tumor sample section may be performed by a trained histologist by microscopy or using a histology slide scanner with automated software analysis. Any of such instruments may need to be properly calibrated to allow expressing the amount of CD8⁺ T-cells per mm² and allowing to compare the computed amount to the predetermined threshold.

In another embodiment, the invention relates to a dendritic cell vaccine for use in a method of treating cancer in a patient, wherein the method comprises a step of determining in a tumor sample of the patient, whether or not the tumor sample has a TMB that is equal or below a predetermined threshold level. The predetermined threshold level may be about 2.5 mut/Mb. In one embodiment, the predetermined threshold level is 2.3 mut/Mb. The inventors have shown that patients with newly diagnosed epithelial ovarian carcinoma, who have undergone debulking surgery and treated with a DC vaccine in parallel or sequentially to standard of care, selected for a TMB in a tumor sample equal or below 2.3 mut/Mb, had an improved progression-free survival (PFS) and overall survival (OS) when compared to patients selected for a TMB in a tumor sample equal or below 2.3 mut/Mb but only treated with the standard of care (see Example 6 below; see also FIG. 7 and FIG. 8).

In one embodiment, the DC vaccine is for the treatment of a tumor. In another embodiment, the invention relates to a DC vaccine for the treatment of cancer, wherein the cancer is ovarian, lung or prostate cancer, preferably ovarian cancer, preferably newly diagnosed ovarian cancer. Ovarian cancer includes epithelial ovarian, fallopian tube, or primary peritoneal cancer and may be stage I to IV according to the International Federation of Gynecology and Obstetrics (FIGO). Prostate cancer includes acinar adenocarcinoma, ductal adenocarcinoma, transitional cell cancer, squamous cell cancer and small cell cancer and may be graded using the Gleason scoring system. Lung cancer is preferentially non-small cell lung cancer and includes lung adenocarcinoma, squamous cell lung carcinoma and large-cell lung carcinoma.

In another embodiment, the tumor sample is obtained prior to the treatment of the patient with the dendritic cell vaccine and an optional chemotherapy, where the chemotherapy is the standard of care.

The standard of care differs for each cancer to be treated and may depend on the stage of the cancer and whether the cancer is newly diagnosed or a recurrent cancer.

In the preferred embodiment of ovarian cancer, the European Society of Medical Oncology (ESMO) established standard of care guidelines of treatment options for patients with newly diagnosed ovarian cancer (Ledermann et al. 2013).
Stage I
   Surgery is the initial method aiming at resection of the tumor and adequate staging of the disease.
   Adjuvant chemotherapy (after completion of surgery):
      Platinum-based chemotherapy with carboplatin
      Combination of carboplatin with paclitaxel
Stage II-IV
   Surgery should be focused on complete cytoreduction of all macroscopic disease. Benefit of systematic pelvic and para-aortic lymphadenectomy is considered questionable at this stage.
   Chemotherapy in all patients (after or before surgery)
      Paclitaxel with carboplatin combination is the standard of care. Carboplatin can be replaced by cisplatin; however, cisplatin is more toxic, and its administration is less convenient.
      In case of paclitaxel toxicity, docetaxel in combination with carboplatin or pegylated liposomal doxorubicin with carboplatin are considered acceptable options.
      Alternative schemes of paclitaxel and carboplatin administration, such as intraperitoneal administration or dense-dose regimens, have also been considered, however they are not supported as a standard of care due to the lack of confirmatory data.
   Targeted therapy: Bevacizumab (biologic, anti-VGEF-A antibody) as an add-on therapy to first-line chemotherapy is indicated for the front-line treatment of adult patients with advanced (International Federation of Gynecology and Obstetrics [FIGO] stages III B, III C, and IV) epithelial ovarian, fallopian tube, or primary peritoneal cancer, followed by continued use of bevacizumab as a single agent until disease progression or for a maximum of 15 months or until unacceptable toxicity, whichever occurs earlier.

In another embodiment, at least one chemotherapeutic agent is selected from carboplatin, cisplatin, paclitaxel, docetaxel, gemcitabine, pegylated liposomal doxorubicin, etoposide, topotecan, irinotecan, olaparib, rucaparib, trabectedin, niraparib, mitoxantrone, cabazitaxel, vinorelbine, pemetrexed, vinblastine, and albumin-bound paclitaxel. The specific regimen of chemotherapy may be dictated by the established standard of care of the specific cancer to be treated and the choice of the treatment may be at the discretion of the practitioner. Chemotherapy may include drugs prescribed as maintenance therapies or prescribed until progression of the cancer such as hormonal therapies (e.g. enzalutamide, abiraterone, tamoxifen, LHRH agonists and antagonists), targeted therapies (e.g. erlotinib, afatinib, gefitinib, crizotinib, alectinib, ceritinib, dabrafenib, trametinib and osimertinib) and biologics (e.g. antibodies as for example bevacizumab, pembrolizumab, nivolumab).

Depending on the standard of care, the chemotherapeutic agent/s may be administered in one or multiple cycles. The duration, frequency and number of cycles will depend on the cancer to be treated and on the agent/s used. The chemotherapeutic agent/s may all be given on a single day, several consecutive days, or continuously, to an outpatient or to an inpatient. Treatment could last minutes, hours, or days, depending on the specific protocol. Chemotherapy may be repeated weekly, bi-weekly, or monthly. Usually, a cycle is defined in monthly intervals. For example, two bi-weekly chemotherapy sessions followed by a recovery period may be classified as one cycle. In most cases, the total number of cycles—or the length of chemotherapy from start to finish—has been determined by research and clinical trials. As long as there are detectable signs of cancer, the length of chemotherapy treatment will depend upon the response of the cancer to therapy. If the cancer disappears completely (complete response), chemotherapy may continue for 1-2 cycles beyond this observation to maximize the chance of having attacked all microscopic, i.e. undetectable, tumors. If the cancer shrinks but does not disappear (partial response), chemotherapy may continue as long as it is tolerated, and the cancer does not grow (progression).

In another embodiment, the amount of CD8⁺ T-cells in the tumor section is measured by immunostaining, preferably immunohistochemistry or immunofluorescence. Immunohistochemistry may be performed as disclosed in Example 2. It is understood that the amount/count of CD8⁺ T-cells in a tumor sample will not be affected by the technique used to visualize the cells. Any antibody binding CD8⁺ T-cells may be used.

In one embodiment, the DC vaccine comprises DCs. In one embodiment, the DC vaccine comprises DCs derived from monocytes that are autologous to the patient to be treated and wherein the monocytes are obtained by leukapheresis.

In another aspect of the invention, the DCs may be derived from monocytes that are autologous to the patient being treated. As used herein, the term “monocytes” refers to leukocytes circulating in the blood characterized by a bean-shaped nucleus and by the absence of granules. Monocytes can give rise to dendritic cells. The monocytes can be isolated from a patient's blood by any technique known to one of skill in the art, the preferred method being leukapheresis. Leukapheresis allows to collect monocytes that are autologous to the patient being treated, to be used for the preparation of the DC vaccine. Leukapheresis may be performed by any technique known to one of skill in the art. Typically, dendritic cells are derived from monocytes obtained by leukapheresis prior to the chemotherapy treatment which may be combined with the DC vaccine treatment.

Different types of DC vaccines are well known in the art and can be divided into different groups according to the method how the DCs are loaded or pulsed with tumor antigens (Turnis and Rooney 2010; Elster, Krishnadas, and Lucas 2016), incorporated herein by reference. Accordingly, the DC vaccine for use in the treatment of cancer administered to a patient is a DC vaccine loaded ex-vivo with an antigen source, which is preferably selected from tumor associated peptide(s), whole antigens from DNA or RNA, whole antigen-protein, idiotype protein, tumor lysate, whole tumor cells or viral vector-delivered whole antigen.

In a preferred embodiment, the immature dendritic cells are in a first step loaded with tumor cells undergoing immunogenic cell death (ICD) and thereafter are matured with Toll-like receptor 3 agonists or Toll-like receptor 4 agonists. ICD, a specific type of apoptosis, may be characterized by expression of immunogenic molecules on the cell surface such as HSP70, HSP90 and calreticulin and the release of late apoptotic markers HMGB1 and ATP. ICD increases the uptake of the apoptotic cells by DCs, resulting in loaded DCs presenting the multiple tumor antigens. In a preferred embodiment of the invention, the antigen source is whole tumor cells and wherein preferably the tumor cells were killed by high hydrostatic pressure (HHP), e.g. as described in WO 2013/004708 and WO 2015/097037, incorporated herein by reference (see examples 1 to 4 of WO 2013/004708 and examples 2 and 3 of WO 2015/097037). In brief, whole tumor cells from cell lines or from the patient to be treated (autologous approach) are treated by HHP between 200 and 300 MPa for 10 min to 2 hours. Such a treatment will induce ICD. Other methods to introduce ICD are the treatment with anthracyclines (Casares et al. 2005) or heat—preferably severe heat above 45° C. (Adkins et al. 2017). Prior to being loaded on DCs, the apoptotic tumor cells may be cryopreserved (see WO 2015/097037).

In one embodiment, the tumor cells loaded upon the DC vaccine are allogeneic to the patient. Whereas autologous tumor cells purportedly have a better match with the patient's tumor antigens, in practice it is highly complicated to manufacture a DC vaccine from autologous tumor biopsies. Therefore, it is preferred to use allogeneic tumor cells, e.g. tumor cells from tumor cell lines, which have an overlap of expressed tumor antigens with the typical tumor antigens of the tumor disease to be treated.

In yet another embodiment, following leukapheresis, the collected autologous monocytes are cultivated in the presence of cytokines to obtain immature DCs. Preferred cytokines are GM-CSF and IL-4. The DC vaccine is obtained by loading the immature DCs with tumor cells undergoing ICD. Preferably, the tumor cells undergoing ICD have been rendered apoptotic by HHP-treatment and are from allogeneic tumor cell lines of the same tumor origin as the one to be treated.

Maturation may be performed with toll-like receptor 3 (TLR3), e.g. poly[I:C], or toll-like receptor 4 (TLR4) agonists, e.g. LPS. Preferentially, the DC vaccine is matured with the TLR3 agonist poly[I:C].

Resulting DC vaccines may be fractioned and stored in individual doses of approximately 1×10⁷ DCs per dose.

Mature DCs generated as described herein may be characterized as displaying significantly higher expression of maturation markers, such as CD80, CD83, HLA-DR and CD86, than immature DCs and DCs loaded with tumor cells killed by other modalities, such as UV irradiation, as shown by Hradilova et al. and Fucikova et al. (Hradilova et al. 2017; Fucikova et al. 2014). Furthermore, Fucikova et al. and Hradilova et al. also have shown that DCs generated as described herein induced a greater number of tumor-specific CD4⁺ and CD8⁺ IFN-γ-producing T cells and decreased the number of CD4⁺CD25⁺Foxp3⁺T regulatory cells compared to DCs pulsed with UV-B light-exposed cells.

The tumor lysate may be autologous or allogeneic/heterologous. Likewise, whole tumor cells may be autologous or allogeneic/heterologous. Whole tumor cells and tumor lysate may originate from one or multiple tumor cell lines and may be selected for a specific presence of antigens matching the patient's tumor to be treated. The source of antigens may be selected and DCs may be loaded by techniques known to one of skill in the art (Turnis and Rooney 2010; Elster, Krishnadas, and Lucas 2016).

The DC vaccine may be formulated for intravenous, intradermal or subcutaneous administrations, preferably for subcutaneous administration. The DC vaccine can be formulated for example for infusion or bolus injection, and may be administered together with an adjuvant (Elster, Krishnadas, and Lucas 2016). Administration can be systemic, loco-regional or local. When a dose is injected, the dose can be administered in one or multiple injections, preferably in two injections. Preferably, the DC vaccine may be administered to the patient subcutaneously into lymph-node regions close to the region where the cancer to be treated is developing. Various delivery systems are known and can be used to deliver the DC vaccine. The mode of administration may be left to the discretion of the practitioner.

In another embodiment, the DC vaccine is administered to a patient in parallel to chemotherapy with at least one chemotherapeutic agent. In another embodiment, the DC vaccine is administered to a patient sequentially to chemotherapy after completion of chemotherapy with at least one chemotherapeutic agent.

When the DC vaccine is administered in parallel to each chemotherapy cycle, it may be further administered after completion/termination of the chemotherapy. The first DC vaccine administration may start with the first cycle of chemotherapy, with the second chemotherapy cycle, with the third chemotherapy cycle, or with a later chemotherapy cycle. Preferentially, the first DC vaccine dose administration starts after the 2^{nd} cycle of chemotherapy and continues after completion/termination of the chemotherapy. In a preferred embodiment, the patient receives at least 6 cycles of chemotherapy.

When the DC vaccine is administered sequentially to chemotherapy after completion of chemotherapy with at least one chemotherapeutic agent, the first dose of the DC vaccine is administered to the patient within two months after administration of the last dose of the chemotherapy, preferably within one month after administration of the last dose of the chemotherapy, more preferably within two weeks after administration of the last dose of the chemotherapy. The first dose of the DC vaccine may be given within the last cycle of chemotherapy, i.e. before completion of the last chemotherapy cycle after administration of the last dose of chemotherapy.

The present invention also relates to a DC vaccine for use in the treatment of a cold tumor.

In one embodiment, the invention relates to a dendritic cell vaccine for use in a method of treating cancer in a patient, wherein the method comprises the step of determining the amount of CD8⁺ T-cells in a tumor sample from the patient and comparing it to a predetermined threshold level.

In one embodiment, the invention relates to a dendritic cell vaccine for use in a method of treating cancer in a patient, wherein the method comprises the step of determining the TMB in a tumor sample from the patient and comparing it to a predetermined threshold level.

In another aspect, the invention relates to a method for selecting a patient for treatment with a dendritic cell vaccine, the method comprising: (i) determining the amount of CD8⁺ T-cells in a tumor sample obtained from the patient, (ii) comparing the amount of CD8⁺ T-cells to a predetermined threshold level, and (iii) selecting the patient for treatment with the dendritic cell vaccine in case the amount of CD8⁺ T-cells in the tumor sample is equal to or below the predetermined threshold level. As cancer treatment often involves a first step of removing most of the tumor mass by debulking surgery, or taking biopsies, a tumor sample of the patient is usually available for evaluation. In one embodiment, the predetermined threshold level is between 30-40 CD8⁺ T-cells/mm². In one embodiment, the predetermined threshold level is about 40 CD8⁺ T-cells/mm², about 35 CD8⁺ T-cells/mm² and preferably about 30 CD8⁺ T-cells/mm². In one embodiment, the method is an in vitro method.

In another aspect, the invention relates to a method for selecting a patient for treatment with a dendritic cell vaccine, the method comprising: (i) determining the TMB in a tumor sample obtained from the patient, (ii) comparing the TMB to a predetermined threshold level, and (iii) selecting the patient for treatment with the dendritic cell vaccine in case the TMB in the tumor sample is equal to or below the predetermined threshold level. The predetermined threshold level may be about 2.5 mut/Mb. In one embodiment, the predetermined threshold level is 2.3 mut/Mb.

In another aspect, the invention relates to a method for determining whether a tumor is responsive to treatment with a dendritic cell vaccine, the method comprising: (i) determining the amount of CD8⁺ T-cells in a tumor sample obtained from a patient, (ii) comparing the amount of CD8⁺ T-cells to a predetermined threshold level, and (iii) selecting the patient for treatment with the dendritic cell vaccine in case the amount of CD8⁺ T-cells in the tumor sample is equal to or below the predetermined threshold level. In one embodiment, the threshold level is between 30-40 CD8⁺ T-cells/mm². In one embodiment, the predetermined threshold level is about 40 CD8⁺ T-cells/mm², about 35 CD8⁺ T-cells/mm² and preferably about 30 CD8⁺ T-cells/mm². In one embodiment, the method is an in vitro method.

In another aspect, the invention relates to a method for determining whether a tumor is responsive to treatment with a dendritic cell vaccine, the method comprising: (i) determining the TMB in a tumor sample obtained from a patient, (ii) comparing the TMB to a predetermined threshold level, and (iii) selecting the patient for treatment with the dendritic cell vaccine in case the TMB in the tumor sample is equal to or below the predetermined threshold level. The predetermined threshold level may be about 2.5 mut/Mb. In one embodiment, the predetermined threshold level is 2.3 mut/Mb.

The invention also involves methods of treatment involving the above described methods for selecting a patient for treatment with a dendritic cell vaccine or for determining whether a tumor is responsive to treatment with a dendritic cell vaccine.

The invention further involves a method of priming a T cell response in a tumor patient, the method comprising: (i) determining the amount of CD8⁺ T-cells in a tumor sample obtained from the patient, (ii) comparing the amount of CD8⁺ T-cells to a predetermined threshold level, (iii) selecting the patient for treatment with the dendritic cell vaccine in case the amount of CD8⁺ T-cells in the tumor sample is equal to or below the predetermined threshold level, and (iv) treating the patient with a dendritic cell vaccine. In one embodiment, the threshold level is between 30-40 CD8⁺ T-cells/mm². In one embodiment, the predetermined threshold level is about 40 CD8⁺ T-cells/mm², about 35 CD8⁺ T-cells/mm² and preferably about 30 CD8⁺ T-cells/mm².

The invention further involves a method of priming a T cell response in a tumor patient, the method comprising: (i) determining the TMB in a tumor sample obtained from the patient, (ii) comparing the TMB to a predetermined threshold level, (iii) selecting the patient for treatment with the dendritic cell vaccine in case the TMB in the tumor sample is equal to or below the predetermined threshold level, and (iv) treating the patient with a dendritic cell vaccine. The predetermined threshold level may be about 2.5 mut/Mb. In one embodiment, the predetermined threshold level is 2.3 mut/Mb.

### DESCRIPTION OF THE DRAWINGS

FIG. 1: Schematic diagram of the clinical study on patients with newly diagnosed ovarian cancer, comparing the standard of care chemotherapy treatment to the DC vaccine treatment administered in parallel or sequentially to chemotherapy. “DCVAC OvCa” stands for a dendritic cell vaccine wherein dendritic cells have been loaded with ovarian cancer cells undergoing immunogenic cell death and matured by a Toll-like receptor ligand. The treatment period 1 corresponds to the period of chemotherapy, also including DC vaccine administration for treatment Group A. The treatment period 2 corresponds to the period when the DC vaccine was administered after completion of the chemotherapy.

FIG. 2: Analysis of progression-free survival (PFS) in the clinical study for the mITT population with newly diagnosed ovarian cancer stratified for the relative count of CD8⁺ T cells/mm² in a tumor tissue section. A) patients with CD8⁺ T cells/mm²≤30; B) patients with CD8⁺ T cells/mm²>30.

FIG. 3: Analysis of overall survival (OS) in the clinical study for the mITT population with newly diagnosed ovarian cancer stratified for the relative count of CD8⁺ T cells/mm² in a tumor tissue section. A) patients with CD8⁺ T cells/mm²≤30; B) patients with CD8⁺ T cells/mm²>30.

FIG. 4: Representative immunohistochemistry microscopy images of ovarian cancer tumor tissue samples showing infiltrated CD8⁺ T-cells. Top panel: tumor sample with an amount of CD8⁺ T-cells/mm² equal or below 30. The black arrow points to stained CD8⁺ T-cells. Bottom panel: tumor sample with an amount of CD8⁺ T-cells/mm² above 30. The black dots are stained CD8⁺ T-cells.

FIG. 5: Distribution of amount of CD8⁺ T-cells/mm² among the mITT population (shows the percent of patient samples containing a specific amount of CD8⁺ T-cells/mm²).
A. Distribution among all the tested samples.
B. Distribution among samples having less than 200 CD8⁺ T-cells/mm².
C. Distribution among samples of amount of CD8⁺ T-cells/mm² for patients treated with DCVAC in parallel to chemotherapy (top), with DCVAC sequentially to chemotherapy (medium) and with standard of care (SoC) alone (bottom).
D. Distribution among samples having less than 200 CD8⁺ T-cells/mm² for patients treated patients with DCVAC in parallel to chemotherapy (top), with DCVAC sequentially to chemotherapy (medium) and with standard of care (SoC) alone (bottom).

FIG. 6: Correlation between the amount of CD8+ T-cells/mm² in ovarian tumor tissue sections and the measured TMB in tumor samples. Upper panel: average amount of CD8+ T-cells/mm² in tumor tissue sections from tumor samples with a TMB≤2.3 mut/Mb (TMB^{Lo}) or a TMB>2.3 mut/Mb (TMB^{Hi}); lower panel: average TMB in tumor samples where CD8⁺ T cells/mm²≤30 in the tumor tissue section (CD8 Low) or CD8⁺ T cells/mm²>30 (CD8 High).

FIG. 7: Analysis of progression-free survival (PFS) in the clinical study for the population with newly diagnosed ovarian cancer stratified for the TMB in the tumor sample. Upper panel: patients with TMB≤2.3 mut/Mb; Lower panel: patients with TMB>2.3 mut/Mb.

FIG. 8: Analysis of overall survival (OS) in the clinical study for the population with newly diagnosed ovarian cancer stratified for the TMB in the tumor sample. Upper panel: patients with TMB≤2.3 mut/Mb; Lower panel: patients with TMB>2.3 mut/Mb.

### THE INVENTION IS ALSO DESCRIBED BY THE FOLLOWING EMBODIMENTS

1. A dendritic cell vaccine for use in a method of treating cancer in a patient, wherein the patient is selected for treatment with said dendritic cell vaccine, the selection comprising determining in a tumor sample from the patient the tumor mutation burden (TMB) and comparing the determined TMB to a predetermined threshold level.
2. The dendritic cell vaccine for use of embodiment 1, wherein the predetermined threshold level is about 2.5 mut/Mb, preferably about 2.3 mut/Mb.
3. The dendritic cell vaccine of for use of embodiment 2, wherein the patient is selected for treatment in case the patient has a TMB equal to or below the predetermined threshold level.
4. A dendritic cell vaccine for use in a method of treating cancer in a patient, wherein the method comprises a step of determining in a tumor sample of the patient, whether or not the patient has a TMB that is equal to or below a predetermined threshold level of about 2.5 mut/Mb, preferably about 2.3 mut/Mb.
5. The dendritic cell vaccine for use of any one of embodiments 1 to 4, wherein the cancer is ovarian, lung or prostate cancer, preferably ovarian cancer, more preferably newly diagnosed ovarian cancer.
6. The dendritic cell vaccine for use of any one of embodiments 1 to 5, wherein the tumor sample is obtained prior to the treatment of the patient with the dendritic cell vaccine.
7. The dendritic cell vaccine for use of embodiment 6, wherein the tumor sample is obtained prior to the treatment of the patient with chemotherapy.
8. The dendritic cell vaccine for use of any one of embodiments 1 to 7, wherein the TMB in the tumor sample is measured by sequencing, preferably next generation sequencing.
9. The dendritic cell vaccine for use of any one of embodiments 1 to 8, wherein the dendritic cells are derived from monocytes that are autologous to the patient to be treated and wherein the monocytes are obtained by leukapheresis.
10. The dendritic cell vaccine for use of any one of embodiments 1 to 9, wherein the dendritic cell vaccine is prepared by loading immature dendritic cells in a first step with tumor cells undergoing immunogenic cell death and thereafter maturing the loaded dendritic cells with Toll-like receptor 3 agonists or Toll-like receptor 4 agonists.
11. The dendritic cell vaccine for use of any one of embodiments 1 to 10, wherein the dendritic cell vaccine is administered to a patient in combination with a further treatment modality selected from the group consisting of chemotherapy, targeted therapy, and biologics.
12. The dendritic cell vaccine for use of any one of embodiments 1 to 11, wherein the dendritic cell vaccine is administered to a patient
   a. in parallel to chemotherapy with at least one chemotherapeutic agent, or
   b. sequentially to chemotherapy after completion of chemotherapy with at least one chemotherapeutic agent.

### The Invention is Further Described by the Following Embodiments

1. A dendritic cell vaccine for use in a method of treating cancer in a patient, wherein the patient is selected for treatment with said dendritic cell vaccine, the selection comprising determining in a tumor sample from the patient the amount of CD8⁺ T-cells and comparing the determined amount to a predetermined threshold level.
2. The dendritic cell vaccine for use of embodiment 1, wherein the amount of CD8⁺ T-cells is determined from at least one section of the tumor sample, the section having a thickness between 2 and 4 μm.
3. The dendritic cell vaccine for use of embodiment 1 or 2, wherein the predetermined threshold level is between about 30-40 CD8⁺ T-cells/mm², preferably about 40 CD8⁺ T-cells/mm², more preferably about 35 CD8⁺ T-cells/mm² and most preferably about 30 CD8⁺ T-cells/mm².
4. The dendritic cell vaccine for use of embodiment 3, wherein the patient is selected for treatment in case the patient has an amount of CD8⁺ T-cells/mm² equal to or below the predetermined threshold level.
5. A dendritic cell vaccine for use in a method of treating cancer in a patient, wherein the method comprises a step of determining in a tumor sample of the patient, whether or not the patient has an amount of CD8⁺ T-cells/mm² that is equal to or below a predetermined threshold level of between about 30-40 CD8⁺ T-cells/mm², preferably about 40 CD8⁺ T-cells/mm², more preferably about 35 CD8⁺ T-cells/mm² and most preferably about 30 CD8⁺ T-cells/mm².
6. The dendritic cell vaccine for use of any one of embodiments 1 to 5, wherein the cancer is ovarian, lung or prostate cancer, preferably ovarian cancer, more preferably newly diagnosed ovarian cancer.
7. The dendritic cell vaccine for use of any one of embodiments 1 to 6, wherein the tumor sample is obtained prior to the treatment of the patient with the dendritic cell vaccine.
8. The dendritic cell vaccine for use of embodiment 7, wherein the tumor sample is obtained prior to the treatment of the patient with chemotherapy.
9. The dendritic cell vaccine for use of any one of embodiments 1 to 8, wherein the amount of CD8⁺ T-cells in the tumor section is measured by immunostaining, preferably immunohistochemistry or immunofluorescence.
10. The dendritic cell vaccine for use of any one of embodiments 1 to 9, wherein the dendritic cells are derived from monocytes that are autologous to the patient to be treated and wherein the monocytes are obtained by leukapheresis.
11. The dendritic cell vaccine for use of any one of embodiments 1 to 10, wherein the dendritic cell vaccine is prepared by loading immature dendritic cells in a first step with tumor cells undergoing immunogenic cell death and thereafter maturing the loaded dendritic cells with Toll-like receptor 3 agonists or Toll-like receptor 4 agonists.
12. The dendritic cell vaccine for use of any one of embodiments 1 to 11, wherein the dendritic cell vaccine is administered to a patient in combination with a further treatment modality selected from the group consisting of chemotherapy, targeted therapy, and biologics.
13. The dendritic cell vaccine for use of any one of embodiments 1 to 12, wherein the dendritic cell vaccine is administered to a patient
   a. in parallel to chemotherapy with at least one chemotherapeutic agent, or
   b. sequentially to chemotherapy after completion of chemotherapy with at least one chemotherapeutic agent.

### The Invention is Additionally Described by the Following Embodiments

1. A dendritic cell vaccine for use in a method of treating a tumor in a patient, wherein the patient is selected for treatment with said dendritic cell vaccine, the selection comprising determining that the tumor is a cold tumor.
2. A dendritic cell vaccine for use in a method of treating a tumor in a patient, wherein the patient is selected for treatment with said dendritic cell vaccine, the selection comprising determining that the tumor exhibits a low amount or the absence of infiltrating CD8⁺ T-cells.
3. The dendritic cell vaccine for use of embodiment 1 or embodiment 2, wherein the selection comprises determining in a tumor sample from the patient the amount of CD8⁺ T-cells and comparing the determined amount to a predetermined threshold level.
4. The dendritic cell vaccine for use of embodiment 3, wherein the amount of CD8⁺ T-cells is determined from at least one section of the tumor sample, the section having a thickness between 2 and 4 μm.
5. The dendritic cell vaccine for use of embodiment 3 or 4, wherein the predetermined threshold level is between about 30-40 CD8⁺ T-cells/mm², preferably about 40 CD8⁺ T-cells/mm², more preferably about 35 CD8⁺ T-cells/mm² and most preferably about 30 CD8⁺ T-cells/mm².
6. The dendritic cell vaccine for use of embodiment 5, wherein the patient is selected for treatment in case the patient has an amount of CD8⁺ T-cells/mm² equal to or below the predetermined threshold level.
7. The dendritic cell vaccine for use of any one of embodiments 1 to 5, wherein the selection comprises determining in a tumor sample from the patient the tumor mutation burden (TMB) and comparing the determined TMB to a predetermined threshold level.
8. The dendritic cell vaccine for use of embodiment 7, wherein the predetermined threshold level is about 2.5 mut/Mb, preferably about 2.3 mut/Mb.
9. The dendritic cell vaccine for use of embodiment 8, wherein the patient is selected for treatment in case the patient has a TMB equal to or below the predetermined threshold level.
10. A dendritic cell vaccine for use in a method of treating cancer in a patient, wherein the method comprises a step of determining in a tumor sample of the patient, whether or not the patient has
   (i) an amount of CD8⁺ T-cells/mm² that is equal to or below a predetermined threshold level of between about 30-40 CD8⁺ T-cells/mm², preferably about 40 CD8⁺ T-cells/mm², more preferably about 35 CD8⁺ T-cells/mm² and most preferably about 30 CD8⁺ T-cells/mm², and/or
   (ii) a TMB that is equal to or below a predetermined threshold level of about 2.5 mut/Mb, preferably about 2.3 mut/Mb.
11. The dendritic cell vaccine for use of any one of embodiments 1 to 10, wherein the cancer is ovarian, lung or prostate cancer, preferably ovarian cancer, more preferably newly diagnosed ovarian cancer.
12. The dendritic cell vaccine for use of any one of embodiments 1 to 11, wherein the tumor sample is obtained prior to the treatment of the patient with the dendritic cell vaccine.
13. The dendritic cell vaccine for use of embodiment 12, wherein the tumor sample is obtained prior to the treatment of the patient with chemotherapy.
14. The dendritic cell vaccine for use of any one of embodiments 3 to 13, wherein the amount of CD8⁺ T-cells in the tumor section is measured by immunostaining, preferably immunohistochemistry or immunofluorescence and/or wherein the TMB in the tumor sample is measured by sequencing, preferably next generation sequencing.
15. The dendritic cell vaccine for use of any one of embodiments 1 to 14, wherein the dendritic cells are derived from monocytes that are autologous to the patient to be treated and wherein the monocytes are obtained by leukapheresis.
16. The dendritic cell vaccine for use of any one of embodiments 1 to 15, wherein the dendritic cell vaccine is prepared by loading immature dendritic cells in a first step with tumor cells undergoing immunogenic cell death and thereafter maturing the loaded dendritic cells with Toll-like receptor 3 agonists or Toll-like receptor 4 agonists.
17. The dendritic cell vaccine for use of any one of embodiments 1 to 16, wherein the dendritic cell vaccine is administered to a patient in combination with a further treatment modality selected from the group consisting of chemotherapy, targeted therapy, and biologics.
18. The dendritic cell vaccine for use of any one of embodiments 1 to 17, wherein the dendritic cell vaccine is administered to a patient
   a. in parallel to chemotherapy with at least one chemotherapeutic agent, or
   b. sequentially to chemotherapy after completion of chemotherapy with at least one chemotherapeutic agent.

### EXAMPLES

### Example 1. DC Vaccine (DCVAC)

The DC vaccine consisted of autologous DCs loaded ex vivo with ovarian cancer cells which were killed by immunogenic cell death and matured by a Toll-like receptor 3 (TLR-3) ligand. DCs were derived from autologous monocytes that were obtained by leukapheresis. Monocytes isolated from the leukapheresis product were cultured in the presence of granulocyte macrophage colony-stimulating factor and interleukin 4 to obtain immature DCs. Immature DCs were loaded with cells of the ovarian cancer cell lines OV-90 and SK-OV-3 (in a ratio of 2:1). Before being added to the DC culture, OV-90 and SK-OV-3 cells were treated with high hydrostatic pressure (HHP) (as described in WO 2013/004708, examples 1-4), which induces immunogenic cell death (Fucikova et al. 2014). The tumor cell-loaded DCs were matured by polyinosinic:polycytidylic acid (poly[I:C]), a TLR-3 ligand.

The final product was cryopreserved in doses of approximately 1×10⁷ DCs per vial in 1 ml of CryoStor CS10 freezing medium containing 10% dimethyl sulfoxide. DC vaccine aliquots were transported to the study sites on dry ice at a temperature below −50° C. Each DC vaccine dose was then thawed and diluted in saline to a final volume of 5 ml. The diluted dose was administered to the patient subcutaneously in two applications: one into the inguinal area and one into the contralateral axillary area (2.5 ml to each of the application sites).

### Example 2: Preparation of Tumor Tissue and Cell Quantification

### Immunohistochemistry

Immunostaining with antibody specific for CD8 was performed according to conventional protocols as published previously (Fucikova et al. 2019; Goc et al. 2014; Truxova et al. 2018). Briefly, tumor specimens were fixed in neutral buffered 10% formalin solution for 24 hours and embedded in paraffin as per standard procedures. The standard sample size was 0.5 cm×1 cm×1 cm. Tissue sections of 3 μm of thickness where cut on a microtome. Tissue sections were deparaffinized, followed by antigen retrieval with Target Retrieval Solution (Leica) in TRIS EDTA at pH 8.0 in a heated water bath (98° C., 30 min). Endogenous peroxidase and alkaline phosphatase were blocked with 3% H₂O₂ and levamisole, respectively for 15 min. Thereafter, sections were treated with protein block (DAKO) for 15 min and incubated with the primary anti-CD8 antibody (clone SP16) (Abcam ab1010500), followed by the revelation of enzymatic activity (EnVision+ Sytem-HRP Labelled Polymer anti-rabbit, DAKO). Sections were counterstained with hematoxylin (DAKO) for 30 sec. Images of whole tumor sections were acquired using a Leica Aperio AT2 scanner (Leica).

### Immunofluorescence

Tumor specimens were fixed in neutral buffered 10% formalin solution and embedded in paraffin as per standard procedures. Immunostaining with the primary anti-CD8 antibody (clone SP16) (Abcam ab1010500) was performed according to conventional protocols. Briefly, tissue sections were deparaffinized and rehydrated descending alcohol series (100, 96, 70, and 50%), followed by antigen retrieval with Target Retrieval Solution (Leica) in EDTA pH 8.0 in preheated water bath (97° C., 30 min). Sections were allowed to cool down to RT for 30 min. Sections were then treated with Signal enhancer (Fisher Thermoscientific) for 30 min and blocking buffer for 60 min. The anti-CD8 antibody was applied for 2 hours at RT. Thereafter, slides were incubated with appropriate fluorophore-labelled secondary antibodies for 1 hour at RT. Finally, sections were treated with TrueBlack® Lipofuscin Autofluorescence Quencher (Biotium) for 30 seconds and mounted with ProLong Gold antifade reagent containing DAPI (Thermo Fisher Scientific). The specificity of the staining was determined using appropriate isotype controls. Images of whole tumor sections were acquired using a Leica Aperio AT2 scanner (Leica).

### Cell Quantification

Infiltration of tumor nests by CD8⁺ T cells was quantified in whole tumor sections with Calopix software (Tribvn) as published previously (Goc et al. 2014; Fucikova et al. 2019). Data are reported as absolute number of positive cells/mm².

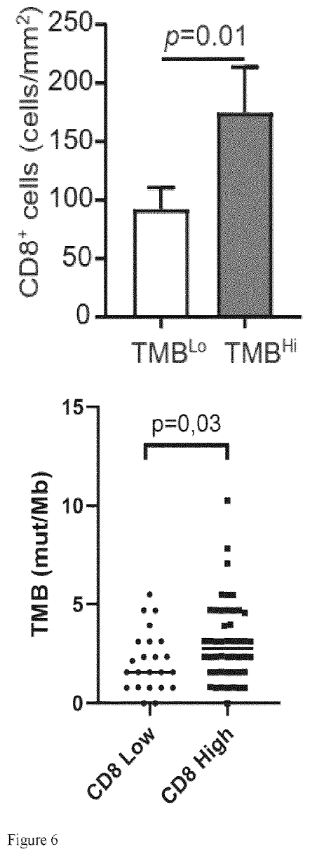

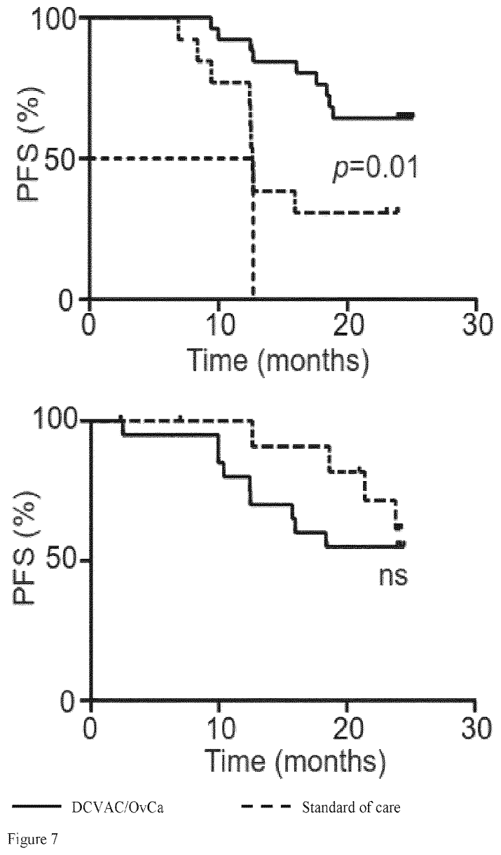

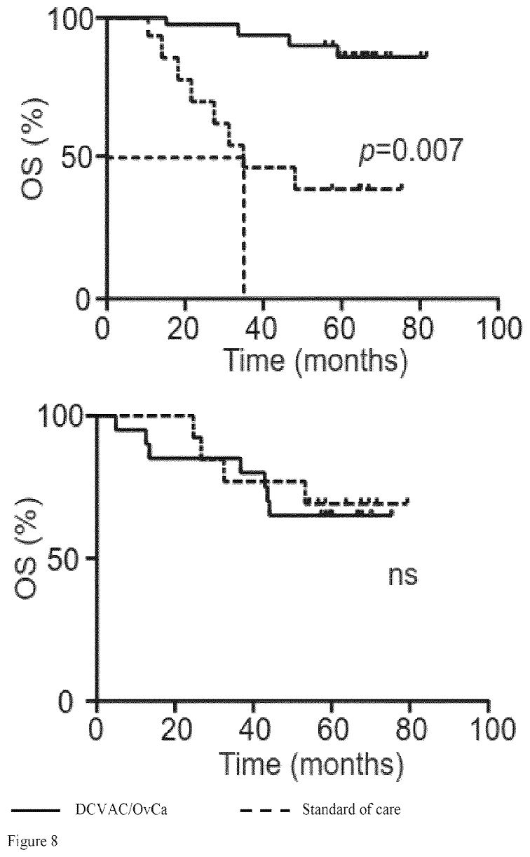

FIG. 4 shows representative immunohistochemistry microscopy images for a patient with a low amount of CD8⁺ T-cells/mm² in the tumor section (4A) and with higher amount of CD8⁺ T-cells/mm² in the tumor section (4B).

### Example 3. Clinical Data of DCVAC Treatment in Combination with Chemotherapy in Women with Newly Diagnosed Epithelial Ovarian Carcinoma

The clinical study (Clinical trial information: NCT02107937, see https://www.clinicaltrials.gov/ct2/show/NCT02107937) was conducted as an open label, multicenter, three-arm phase II clinical trial in women with newly diagnosed epithelial ovarian carcinoma, who have undergone debulking surgery. The aim of this study was to evaluate the efficacy and safety of the DC vaccine (DCVAC) administered in parallel to chemotherapy as an add-on to standard of care chemotherapy with carboplatin and paclitaxel or sequentially after standard of care chemotherapy with carboplatin and paclitaxel compared to chemotherapy alone.

A total of 99 patients were centrally randomized in a ratio of 1:1:1 to treatment groups
A (34 patients) to receive the DC vaccine in parallel with standard of care chemotherapy,
B (34 patients) to receive the DC vaccine sequentially after standard of care chemotherapy, or
C (31 patients) to receive standard of care chemotherapy alone.

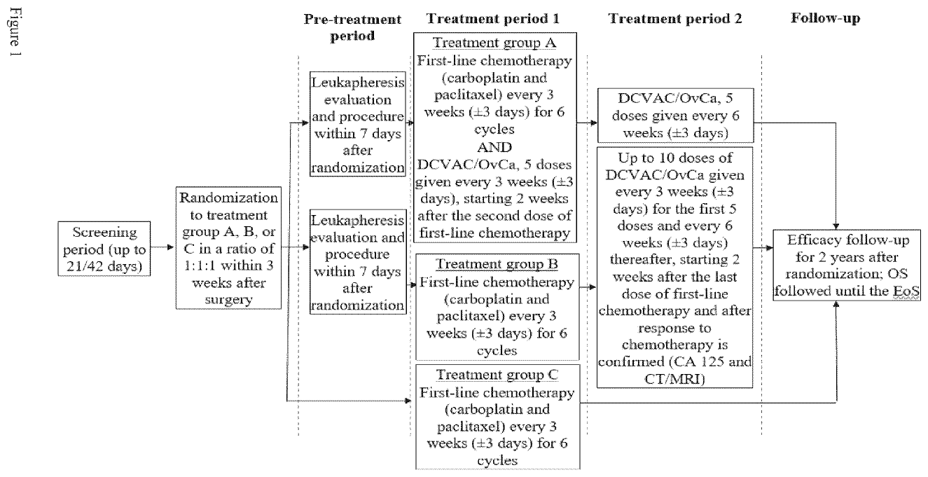

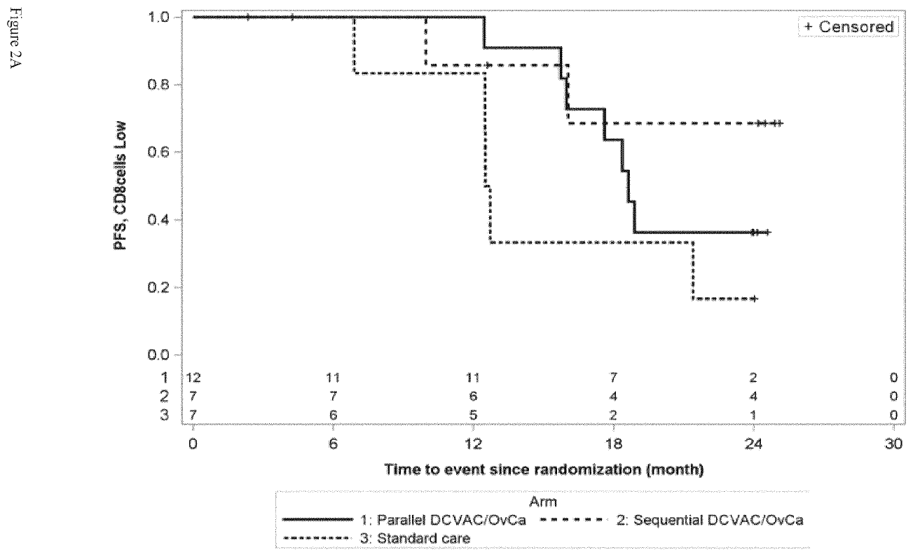

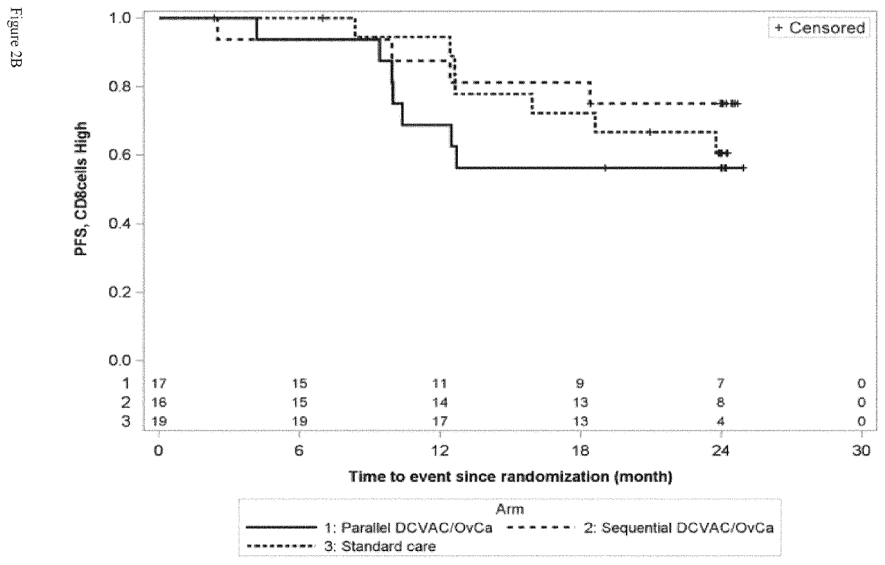

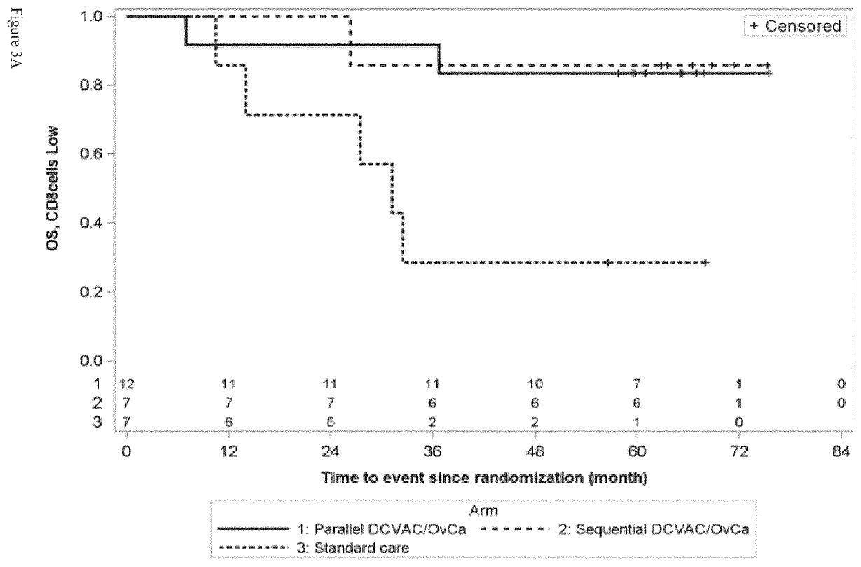

The DC vaccine was administered subcutaneously (SC) to patients in treatment groups A and B in up to 10 doses. The planned number of chemotherapy cycles was 6 in all treatment groups (FIG. 1).

The main population, modified intent-to-treat (mITT) population included all patients who were randomized and received at least 1 dose of chemotherapy in group C, or at least 1 dose of DC vaccine in groups A and B (mITT: 31 patients in treatment group A [parallel DC vaccine], 29 patients in treatment group B [sequential DC vaccine], and 30 patients in treatment group C [standard of care]).

Analysis of Subgroup of Patients with CD8⁺ Low Cells Counts

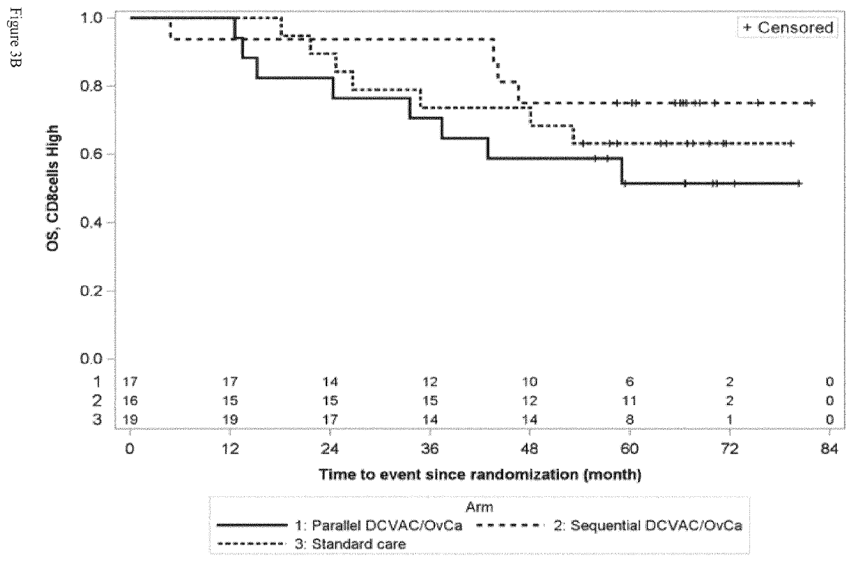

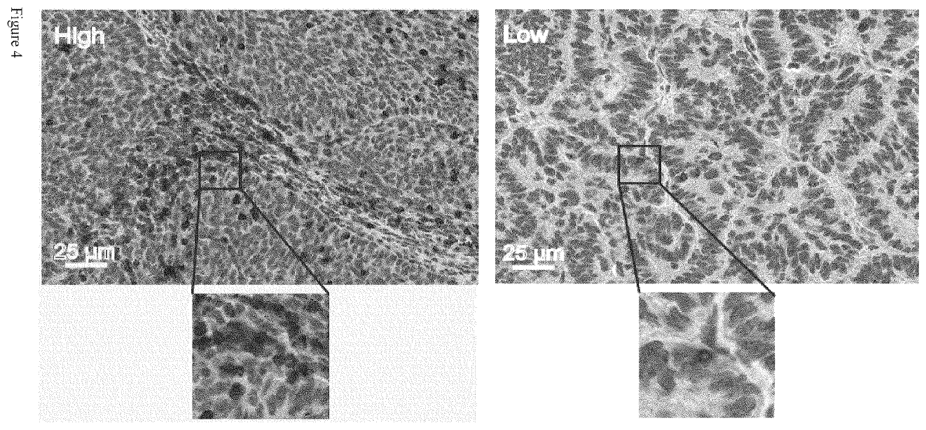

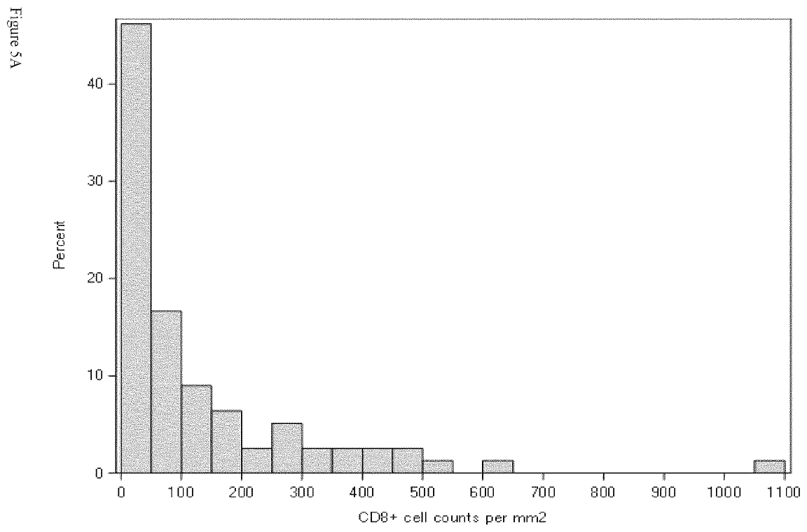

Both PFS and OS were analyzed on the subgroup of patients with low CD8⁺ cells counts (CD8⁺ cells counts ≤30 cells/mm²) (see FIG. 5 on the distribution of CD8⁺ T-cells amounts in a tumor sample over the mITT population). When the count of CD8⁺ T-cells was at below or equal to 30 cells/mm², in the mITT population, group A and B patient had a better PFS when compared to group C patients treated with the standard of care treatment (A vs C: HR=0.49, p=0.2219; B vs C: HR=0.25, p=0.0759). (Table 1, FIG. 2A). That difference in PFS was not seen for patients with count of CD8⁺ T-cells above 30 cells/mm² in the tumor sample when comparing treatment groups A and B to C with standard of care in mITT population (A vs C: HR=1.37, p=0.5518; B vs C: HR=0.65, p=0.4908) (Table 1, FIG. 2B).

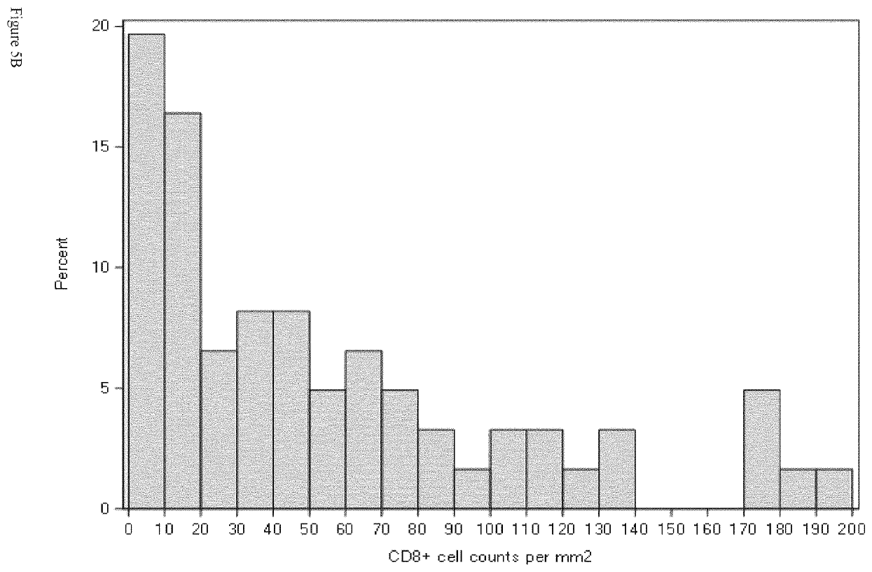

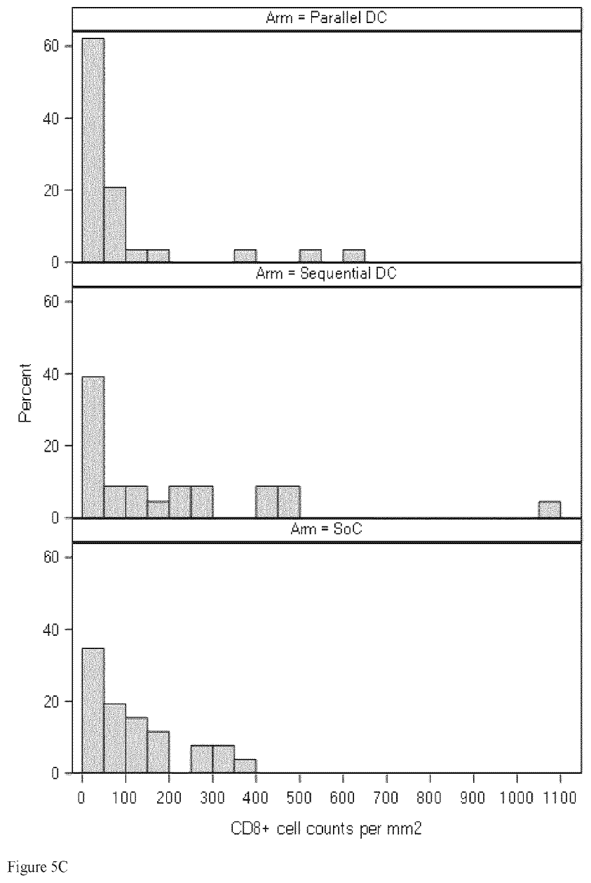

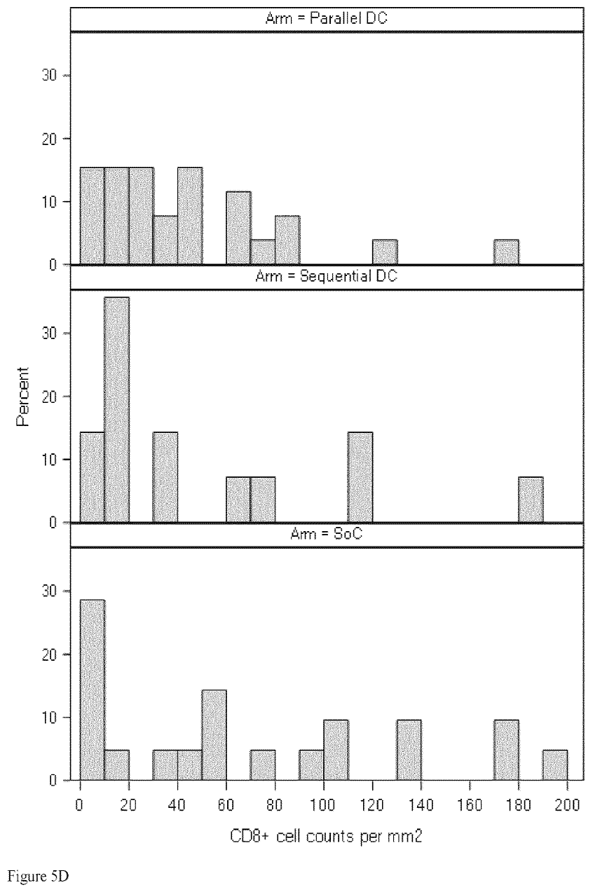

When the count of CD8⁺ T-cells was at below or equal to 30 cells/mm², in the mITT population, group A and B patient had a better OS when compared to group C patients treated with the standard of care treatment (A vs C: HR=0.15, p=0.0131; B vs C: HR=0.15, p=0.0434) (Table 1, FIG. 3A). Again, that difference in OS was not seen for patients with count of CD8⁺ T-cells above 30 cells/mm² in the tumor sample when comparing treatment groups A and B to C with standard of care in mITT population (A vs C: HR=1.41, p=0.5084; B vs C: HR=0.63, p=0.4516) (Table 1, FIG. 3B).

TABLE 1
Benefit of DCVAC/OvCa on patient progression-free survival and overall survival

| | Treatment group A | | Treatment group B | |
|---|---|---|---|---|
| | (parallel) over | | (sequential) over | |
| | treatment group C (SoC) | | treatment group C (SoC) | |
| | HR | p-value | HR | p-value |
| PFS on mITT population and | 0.49 | 0.2219 | 0.25 | 0.0759 |
| CD8⁺ count ≤30 cells/mm² | | | | |
| PFS on mITT population and | 1.37 | 0.5518 | 0.65 | 0.4908 |
| CD8⁺ count >30 cells/mm² | | | | |
| OS on mITT population and | 0.15 | 0.0131 | 0.15 | 0.0434 |
| CD8⁺ count ≤30 cells/mm² | | | | |
| OS on mITT population and | 1.41 | 0.5084 | 0.63 | 0.4516 |
| CD8⁺ count >30 cells/mm² | | | | |

PFS: progression-free survival;
OS: overall survival;
SoC: standard of care;
HR: hazard ratio

### Example 4: Preparation of Tumor RNA and Quantification of the TMB

DNA/RNA Isolation from FFPE

RNA and DNA were isolated using AllPrep DNA/RNA FFPE Kit (Qiagen) according to the manufacturers' instructions. The RNA concentration and purity were determined using a NanoDrop 2000c (Thermo Scientific, Germany). Purified RNA samples were stored at −80° C. until further use.

### DNA Library

DNA libraries were prepared using the hybrid capture-based TruSight Oncology 500 Library Preparation Kit (Illumina, San Diego, CA, USA). TMB measurement and analyses were performed as described in the TruSight Oncology 500 Reference Guide (“TruSight Oncology 500 Reference Guide” 2020)

### Example 5: TMB Status Positively Correlates with Anti-Tumor Immunity in Ovarian Carcinoma

Of the 90 patients enrolled and randomly assigned in the clinical study (Clinical trial information: NCT02107937, see https://www.clinicaltrials.gov/ct2/show/NCT02107937), 78 (86%) had tumor samples available to attempt the assessment of the tumor mutational burden (TMB) and had valid data for TMB-based efficacy analyses. Baseline characteristics of all randomly assigned patients and patients whose TMB could be evaluated were similar and balanced between the SOC treatment group and the DCVAC treatment group. The TrueSightOnco500 gene panel was used to compare the profile of somatic mutations and TMB in tumor samples from ovarian cancer patients involved in the study. A panel of 20 somatic mutations (TP53, BRCA1, BARD1, MDC1, PIK3CA, SDHA, TET1, ARID5B, KRAS, ZFHX3, ZNF703, RID1A, BOOR, BCORL1, FAWCD2, GNAS, MED12, NF1, SLX4 and SPTA1) was identified as occurring in patients and was well balanced between treatment groups. Similarly, a statistical difference between the TMB load in individual patients' treatment arms was not observed. The relationship between the TMB status and intratumoral abundance of CD8⁺ T-cells measured by immunofluorescence was analyzed. A significantly higher densities of CD8⁺ T-cells in tumor samples was observed in samples from TMB^{Hi} patients, compared to TMB^{Lo} patients (FIG. 6, upper panel). Likewise, patients with a low TMB had low CD8⁺ T-cells infiltration in tumor samples, compared to patients with high TMB (see FIG. 6 lower panel). Taken together, these findings indicate that a high TMB status in the tumor microenvironment of ovarian cancer were strongly associated with an immune infiltration and low TMB is associated with poor immune infiltration.

### Example 6: Low Tumor Mutational Burden Status is Associated with Better Clinical Response to DCVAC Therapy

To assess the prognostic and predictive value of TMB in the tumor samples of ovarian patients involved in the clinical study NCT02107937, PFS and OS was evaluated upon median stratification. The median value calculated from the measured TMB on all patient tumor sample was equal to 2.3 mut/Mb. When the patients group was selected for having a TMB in the tumor sample below or equal to 2.3 mut/Mb, the patient group treated with DCVAC had a better PFS and OS compared to patients treated with standard of care only (PFS: FIG. 7, upper panel; OS: FIG. 8, upper panel). When the patients group was selected for having a TMB in the tumor sample above 2.3 mut/Mb, no significant difference in PFS and OS was observed between the patient group treated DCVAC and the patient group treated with standard of care alone (PFS: FIG. 7, lower panel; OS: FIG. 8, lower panel).

### REFERENCES

Adkins, Irena, Lenka Sadilkova, Nada Hradilova, Jakub Tomala, Marek Kovar, and Radek Spisek. 2017. ‘Severe, but not mild heat-shock treatment induces immunogenic cell death in cancer cells’, Oncoimmunology, 6: e1311433.
‘American Cancer Society—How Chemotherapy Drugs Work’. 2016. American Cancer Society, Accessed 2018 Jan. 5. https://www.cancer.org/treatment/treatments-and-side-effects/treatment-types/chemotherapy/how-chemotherapy-drugs-work.html.
Banchereau, J., and R. M. Steinman. 1998. ‘Dendritic cells and the control of immunity’, Nature, 392: 245-52.
Bloy, N., J. Pol, F. Aranda, A. Eggermont, I. Cremer, W. H. Fridman, J. Fucikova, J. Galon, E. Tartour, R. Spisek, M. V. Dhodapkar, L. Zitvogel, G. Kroemer, and L. Galluzzi. 2014. ‘Trial watch: Dendritic cell-based anticancer therapy’, Oncoimmunology, 3: e963424.
Buttner, R., J. W. Longshore, F. Lopez-Rios, S. Merkelbach-Bruse, N. Normanno, E. Rouleau, and F. Penault-Llorca. 2019. ‘Implementing TMB measurement in clinical practice: considerations on assay requirements’, ESMO Open, 4: e000442.
Casares, N., M. O. Pequignot, A. Tesniere, F. Ghiringhelli, S. Roux, N. Chaput, E. Schmitt, A. Hamai, S. Hervas-Stubbs, M. Obeid, F. Coutant, D. Metivier, E. Pichard, P. Aucouturier, G. Pierron, C. Garrido, L. Zitvogel, and G. Kroemer. 2005. ‘Caspase-dependent immunogenicity of doxorubicin-induced tumor cell death’, J Exp Med, 202: 1691-701.
Cibula, D., P. Mallmann, P. Knapp, B. Melichar, J. Klat, L. Minar, Z. Novotny, P. Wimberger, A. Hein, R. Spisek, J. Bartunkova, L. Pecen, H. I. B. Hassan, L. Rob, and SOV02 Investigators. 2018. ‘Dendritic cell vaccine (DCVAC) with chemotherapy (ct) in patients (pts) with recurrent epithelial ovarian carcinoma (EOC) after complete response (CR) to 1st-line platinum (Pt)-based ct: Primary analysis of a phase 2, open-label, randomized, multicenter trial’, Journal of Clinical Oncology, 36: e17515-e15.
Dey, Pranab. 2018. Basic and Advanced Laboratory Techniques in Histopathology and Cytology (Springer Nature Singapore: Singapore).
Ellis, P. M. 2016. ‘Anti-angiogenesis in Personalized Therapy of Lung Cancer’, Adv Exp Med Blot, 893: 91-126.
Elster, J. D., D. K. Krishnadas, and K. G. Lucas. 2016. ‘Dendritic cell vaccines: A review of recent developments and their potential pediatric application’, Hum Vaccin Immunother, 12: 2232-9.
Fridman, W. H., L. Zitvogel, C. Sautes-Fridman, and G. Kroemer. 2017. ‘The immune contexture in cancer prognosis and treatment’, Nat Rev Clin Oncol, 14: 717-34.
Fucikova, J., I. Moserova, I. Truxova, I. Hermanova, I. Vancurova, S. Partlova, A. Fialova, L. Sojka, P. F. Cartron, M. Houska, L. Rob, J. Bartunkova, and R. Spisek. 2014. ‘High hydrostatic pressure induces immunogenic cell death in human tumor cells’, Int J Cancer, 135: 1165-77.
Fucikova, J., J. Rakova, M. Hensler, L. Kasikova, L. Belicova, K. Hladikova, I. Truxova, P. Skapa, J. Laco, L. Pecen, I. Praznovec, M. J. Halaska, T. Brtnicky, R. Kodet, A. Fialova, J. Pineau, A. Gey, E. Tartour, A. Ryska, L. Galluzzi, and R. Spisek. 2019. ‘TIM-3 Dictates Functional Orientation of the Immune Infiltrate in Ovarian Cancer’, Clin Cancer Res, 25: 4820-31.
Galluzzi, L., L. Senovilla, E. Vacchelli, A. Eggermont, W. H. Fridman, J. Galon, C. Sautes-Fridman, E. Tartour, L. Zitvogel, and G. Kroemer. 2012. ‘Trial watch: Dendritic cell-based interventions for cancer therapy’, Oncoimmunology, 1: 1111-34.
Galon, Jerome, and Daniela Bruni. 2019. ‘Approaches to treat immune hot, altered and cold tumours with combination immunotherapies’, Nature Reviews Drug Discovery, 18: 197-218.
Garg, A. D., M. Vara Perez, M. Schaaf, P. Agostinis, L. Zitvogel, G. Kroemer, and L. Galluzzi. 2017. ‘Trial watch: Dendritic cell-based anticancer immunotherapy’, Oncoimmunology, 6: e1328341.
Goc, J., C. Germain, T. K. Vo-Bourgais, A. Lupo, C. Klein, S. Knockaert, L. de Chaisemartin, H. Ouakrim, E. Becht, M. Alifano, P. Validire, R. Remark, S. A. Hammond, I. Cremer, D. Damotte, W. H. Fridman, C. Sautes-Fridman, and M. C. Dieu-Nosjean. 2014. ‘Dendritic cells in tumor-associated tertiary lymphoid structures signal a Th1 cytotoxic immune contexture and license the positive prognostic value of infiltrating CD8+ T cells’, Cancer Res, 74: 705-15.
Head, S R. 2018. Next Generation Sequencing (Humana Press).
Hradilova, N., L. Sadilkova, O. Palata, D. Mysikova, H. Mrazkova, R. Lischke, R. Spisek, and I. Adkins. 2017. ‘Generation of dendritic cell-based vaccine using high hydrostatic pressure for non-small cell lung cancer immunotherapy’, PLoS One, 12: e0171539.
Ledermann, J. A., F. A. Raja, C. Fotopoulou, A. Gonzalez-Martin, N. Colombo, C. Sessa, and Esmo Guidelines Working Group. 2013. ‘Newly diagnosed and relapsed epithelial ovarian carcinoma: ESMO Clinical Practice Guidelines for diagnosis, treatment and follow-up’, Ann Oncol, 24 Suppl 6: vi24-32.
Lisi, L., G. M. Ciotti, D. Braun, S. Kalinin, D. Curro, C. Dello Russo, A. Coli, A. Mangiola, C. Anile, D. L. Feinstein, and P. Navarra. 2017. ‘Expression of iNOS, CD163 and ARG-1 taken as M1 and M2 markers of microglial polarization in human glioblastoma and the surrounding normal parenchyma’, Neurosci Lett, 645: 106-12.
Maleki Vareki, S. 2018. ‘High and low mutational burden tumors versus immunologically hot and cold tumors and response to immune checkpoint inhibitors’, J Immunother Cancer, 6: 157.
Palucka, K., and J. Banchereau. 2013. ‘Dendritic-cell-based therapeutic cancer vaccines’, Immunity, 39: 38-48.
Rob, L., P. Mallmann, P. Knapp, B. Melichar, J. Klat, L. Minar, Z. Novotny, J. Bartunkova, R. Spisek, L. Pecen, H. I. B. Hassan, D. Cibula, and SOV01 Investigators. 2018. ‘Dendritic cell vaccine (DCVAC) with chemotherapy (ct) in patients (pts) with epithelial ovarian carcinoma (EOC) after primary debulking surgery (PDS): Interim analysis of a phase 2, open-label, randomized, multicenter trial’, Journal of Clinical Oncology, 36: 5509-09.
Sakarya, D. K.; Yetimalar, M. H. 2016. ‘Is there a current change of maintenance treatment in ovarian cancer? An updated review of the literature’, JBUON, 21: 290-300.
Schreibelt, G., J. Tel, K. H. Sliepen, D. Benitez-Ribas, C. G. Figdor, G. J. Adema, and I. J. de Vries. 2010. ‘Toll-like receptor expression and function in human dendritic cell subsets: implications for dendritic cell-based anti-cancer immunotherapy’, Cancer Immunol Immunother, 59: 1573-82.
Steele, K. E., T. H. Tan, R. Korn, K. Dacosta, C. Brown, M. Kuziora, J. Zimmermann, B. Laffin, M. Widmaier, L. Rognoni, R. Cardenes, K. Schneider, A. Boutrin, P. Martin, J. Zha, and T. Wiestler. 2018. ‘Measuring multiple parameters of CD8+ tumor-infiltrating lymphocytes in human cancers by image analysis’, J Immunother Cancer, 6: 20.
“TruSight Oncology 500 Reference Guide.” In. 2020. edited by Inc. Illumina, 50. Illumina, Inc.
Truxova, I., L. Kasikova, M. Hensler, P. Skapa, J. Laco, L. Pecen, L. Belicova, I. Praznovec, M. J. Halaska, T. Brtnicky, E. Salkova, L. Rob, R. Kodet, J. Goc, C. Sautes-Fridman, W. H. Fridman, A. Ryska, L. Galluzzi, R. Spisek, and J. Fucikova. 2018. ‘Mature dendritic cells correlate with favorable immune infiltrate and improved prognosis in ovarian carcinoma patients’, J Immunother Cancer, 6: 139.
Turnis, M. E., and C. M. Rooney. 2010. ‘Enhancement of dendritic cells as vaccines for cancer’, Immunotherapy, 2: 847-62.
Vacchelli, E., I. Vitale, A. Eggermont, W. H. Fridman, J. Fucikova, I. Cremer, J. Galon, E. Tartour, L. Zitvogel, G. Kroemer, and L. Galluzzi. 2013. ‘Trial watch: Dendritic cell-based interventions for cancer therapy’, Oncoimmunology, 2: e25771.
WO 2013/004708
WO 2015/097037

1. A dendritic cell vaccine for use in a method of treating a tumor in a patient, wherein the patient is selected for treatment with said dendritic cell vaccine, the selection comprising determining that the tumor is a cold tumor.

2. A dendritic cell vaccine for use in a method of treating a tumor in a patient, wherein the patient is selected for treatment with said dendritic cell vaccine, the selection comprising determining that the tumor exhibits a low amount or the absence of infiltrating CD8⁺ T-cells.

3. The dendritic cell vaccine for use of claim 1 or claim 2, wherein the selection comprises determining in a tumor sample from the patient the amount of CD8⁺ T-cells and comparing the determined amount to a predetermined threshold level, optionally wherein the amount of CD8⁺ T-cells is determined from at least one section of the tumor sample, the section having a thickness between 2 and 4 μm.

4. The dendritic cell vaccine for use of claim 3, wherein the predetermined threshold level is between about 30-40 CD8⁺ T-cells/mm², preferably about 40 CD8⁺ T-cells/mm², more preferably about 35 CD8⁺ T-cells/mm² and most preferably about 30 CD8⁺ T-cells/mm², optionally wherein the patient is selected for treatment in case the patient has an amount of CD8⁺ T-cells/mm² equal to or below the predetermined threshold level.

5. The dendritic cell vaccine for use of any one of claims 1 to 4, wherein the selection comprises determining in a tumor sample from the patient the tumor mutation burden (TMB) and comparing the determined TMB to a predetermined threshold level, optionally wherein the predetermined threshold level is about 2.5 mut/Mb, preferably about 2.3 mut/Mb.

6. The dendritic cell vaccine for use of claim 5, wherein the patient is selected for treatment in case the patient has a TMB equal to or below the predetermined threshold level.

7. A dendritic cell vaccine for use in a method of treating cancer in a patient, wherein the method comprises a step of determining in a tumor sample of the patient, whether or not the patient has
(i) an amount of CD8⁺ T-cells/mm² that is equal to or below a predetermined threshold level of between about 30-40 CD8⁺ T-cells/mm², preferably about 40 CD8⁺ T-cells/mm², more preferably about 35 CD8⁺ T-cells/mm² and most preferably about 30 CD8⁺ T-cells/mm², and/or
(ii) a TMB that is equal to or below a predetermined threshold level of about 2.5 mut/Mb, preferably about 2.3 mut/Mb.

8. The dendritic cell vaccine for use of any one of claims 1 to 7, wherein the cancer is ovarian, lung or prostate cancer, preferably ovarian cancer, more preferably newly diagnosed ovarian cancer.

9. The dendritic cell vaccine for use of any one of claims 1 to 8, wherein the tumor sample is obtained prior to the treatment of the patient with the dendritic cell vaccine.

10. The dendritic cell vaccine for use of claim 9, wherein the tumor sample is obtained prior to the treatment of the patient with chemotherapy.

11. The dendritic cell vaccine for use of any one of claims 3 to 10, wherein the amount of CD8⁺ T-cells in the tumor section is measured by immunostaining, preferably immunohistochemistry or immunofluorescence and/or wherein the TMB in the tumor sample is measured by sequencing, preferably next generation sequencing.

12. The dendritic cell vaccine for use of any one of claims 1 to 11, wherein the dendritic cells are derived from monocytes that are autologous to the patient to be treated and wherein the monocytes are obtained by leukapheresis.

13. The dendritic cell vaccine for use of any one of claims 1 to 12, wherein the dendritic cell vaccine is prepared by loading immature dendritic cells in a first step with tumor cells undergoing immunogenic cell death and thereafter maturing the loaded dendritic cells with Toll-like receptor 3 agonists or Toll-like receptor 4 agonists.

14. The dendritic cell vaccine for use of any one of claims 1 to 13, wherein the dendritic cell vaccine is administered to a patient in combination with a further treatment modality selected from the group consisting of chemotherapy, targeted therapy, and biologics.

15. The dendritic cell vaccine for use of any one of claims 1 to 14, wherein the dendritic cell vaccine is administered to a patient
a. in parallel to chemotherapy with at least one chemotherapeutic agent, or
b. sequentially to chemotherapy after completion of chemotherapy with at least one chemotherapeutic agent.
